(19) United States
(12) Patent Application Publication
(10) Pub. No.: US 2025/0361501 A1
(43) Pub. Date: Nov. 27, 2025

(54) METHODS FOR MINING STRAINS AND GENES, AND USES THEREOF

(71) Applicant: Wuhan Ruijiakang Biotechnology Co., Ltd, Wuhan (CN)

(72) Inventors: Shihui YANG, Wuhan (CN); Weiwei BAO, Wuhan (CN); Qiqun PENG, Wuhan (CN)

(73) Assignee: Wuhan Ruijiakang Biotechnology Co., Ltd, Wuhan (CN)

(21) Appl. No.: 18/671,938

(22) Filed: May 22, 2024

(51) Int. Cl. C12N 15/10; C12N 1/20; C12N 15/66; C12P 7/56; C12R 1/01

(52) U.S. Cl. CPC C12N 15/1034; C12N 1/205; C12N 15/66; C12P 7/56; C12R 2001/01

(57) ABSTRACT

Method for mining strains and genes, and use are provided. The method includes using a recombinant plasmid, and transferring the plasmid into a strain to be mined. The recombinant plasmid carries an operon for catabolizing lactic acid and a gene for report. A use of ZMO1323 mining by the method in biosynthesis of D-LA is provided.

### SEQUENCE LISTING

The sequence listing xml file submitted herewith, named “Sequence_Listing.xml”, created on May 22, 2024, and having a file size of 36,000 bytes, is incorporated by reference herein.

### TECHNICAL FIELD

This disclosure relates to Zymomonas mobilis. Specifically, this disclosure relates to a method for mining strains, a method for mining genes, and uses thereof.

### BACKGROUND

The statements herein provide background information relevant to the present disclosure only and do not necessarily constitute prior art.

As a natural and facultative anaerobic Gram-negative bacterium for producing alcohol, Zymomonas mobilis (Z. mobilis) has a unique ED metabolic pathway and a high efficiency of sugar fermentation, and also has many unique physiological and excellent industrial characteristics, such as few byproducts, high specific productivity of alcohol, high alcohol tolerance, resistant to high osmotic pressure, no need of additional oxygen in the fermentation process. So far, the production of PHB, 2,3-butanediol, isobutanol and lactic acid have been achieved in Z. mobilis. Additionally, the production of ethanol from cellulose has been commercialized in Z. mobilis because of its high tolerance to lignocellulosic hydrolysate, and the study of mechanism for this tolerance is also mature. And, by means of synthetic biology and metabolic engineering, Z. mobilis could be modified into chassis cells that be able to use lignocellulosic hydrolysates for producing different platform compounds.

D-lactic acid (D-LA) is an important monomer in the synthesis of poly lactic acid and petrochemical plastics. In order to obtain a high yield of D-LA to meet the relevant commercial requirements, the mining of microorganisms with high yield of D-LA is an essential means to improve the titer of D-LA except the construction of metabolic pathways. It is of great significance to mine suitable target genes or genetic engineering elements, and construct them into Z. mobilis to achieve efficient production of D-LA.

However, there are many genes involved in synthesis of lactic acid, especially a large number of prokaryotic exogenous genes involved in lactic acid synthesis and expression. Therefore, it is particularly necessary to mine suitable target genes and suitable engineering strains of Z. mobilis.

### SUMMARY

In one aspect, embodiments disclose a method for mining strains to produce D-LA. The method includes: obtaining a recombinant plasmid; transferring the recombinant plasmid into a strain to be mined; and screening positive colonies according to the fluorescence intensity of the strain to be mined. And the strains to produce D-LA could be obtained. The recombinant plasmid carries a connector consisting of a operon for catabolizing D-lactic acid and a gene for report. The operon for catabolizing D-lactic acid is shown as SEQ ID NO. 1.

In another aspect, embodiments disclose a method for mining genes to regulate biosynthesis of D-LA. The method includes: obtaining a recombinant plasmid; transferring the recombinant plasmid to a strain to be mined; screening positive colonies according to the fluorescence intensity of the strain to be mined; and mining the genes to regulate biosynthesis of D-lactic acid from the positive colonies. The recombinant plasmid carries a connector consisting of a operon for catabolizing D-lactic acid and a gene for report, the operon for catabolizing D-lactic acid is shown as SEQ ID NO. 1.

In another aspect, embodiments disclose a use of ZMO1323 in biosynthesis of D-LA. The use includes: preparing an editing plasmid for knocking out the ZMO1323; transferring the editing plasmid into a D-lactic acid production strain of Zymomonas mobilis. The ZMO1323 is locate at CP023715.1:1342357 . . . 1343994 of Zymomonas mobilis subsp. mobilis ZM4=ATCC 31821.

### BRIEF DESCRIPTION OF DRAWINGS

FIG. 1 shows the structure and principle of the biosensor for recognizing D-LA provided with embodiments.

FIG. 2A shows the fluorescence intensity map of the positive colonies with the biosensor on different contents of D-LA in culture provided with embodiments.

FIG. 2B shows the correlation diagram of the positive colonies with the biosensor on different contents of D-LA in culture provided with embodiments.

FIG. 3A shows the step of the method for mining genes to regulate biosynthesis of D-LA consisting of the CRISPRi library (69,093 pEZ-sgr-lib) provided with embodiments.

FIG. 3B shows the step of the method for mining genes to regulate biosynthesis of D-LA consisting of transferring pEZ-sgr-lib into ZM4-dCas12a-Dldh provided with embodiments.

FIG. 3C shows the step of the method for mining genes to regulate biosynthesis of D-LA consisting of screening from the strain library of high throughput screening platform by two rounds of FACS to get mutants provided with embodiments.

FIG. 3D shows the step of the method for mining genes to regulate biosynthesis of D-LA consisting of cultivating and spreading the mutants on RMG5 supplemented with calcium carbonate provided with embodiments.

FIG. 3E shows the step of the method for mining genes to regulate biosynthesis of D-LA consisting of fermenting the mutants in liquid medium to determine D-lactic acid yield provided with embodiments.

FIG. 3F shows the results of mutants' D-lactic acid yield provided with embodiments.

FIG. 3G shows a diagram of Sanger Sequencing for the crRNA sequences in mutants with increased D-lactic acid production provided with embodiments.

FIG. 3H shows a diagram of D-lactic acid production strains based on the identified genes provided with embodiments.

### DETAILED DESCRIPTION OF THE EMBODIMENTS

Embodiments provide a biosensor, a method for mining strains, a method for mining genes, and uses thereof. The biosensor is a recombinant plasmid of pEZ15A that carries an operon for catabolizing lactic acid and a gene for report. By transferring this biosensor into the strain to be mined, the synthesis amount of D-LA could be correlated with the expression of the gene for report. And the strains with increased D-LA production could be effectively mined according to the signal (such as fluorescence signal) generated by the expression of the gene for report. And the genes to regulate the expressing of D-LA could also be effectively mined.

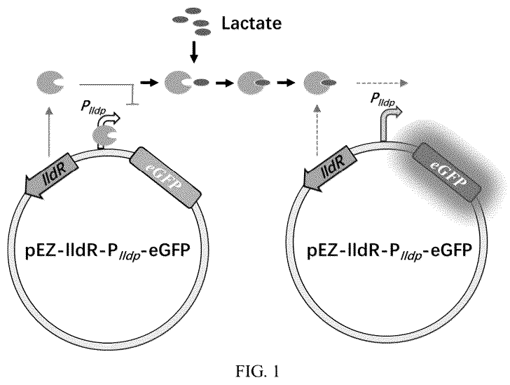

As FIG. 1, embodiments disclose a recombinant plasmid of pEZ15A that carries an operon (shown as SEQ ID NO. 1) for catabolizing lactic acid and a gene for report. The operon includes a gene named lldR and a promoter named P_{lldp}. The gene for report is promoted by P_{lldp}. And the operon could inhibit the expression of the gene for report. However, this inhibition could be mitigated in the context of D-LA, thereby positively correlating the signal generated by the gene for report with the presence and concentration of D-LA. In some embodiments, the transcription direction of the operon is opposite to that of the gene for report.

In some embodiments, the gene for report may be selected from the group consisting of genes of firefly luciferase, marine coelenteric luciferase, secretory alkaline phosphatase (SEAP), human growth hormone (hGH), green fluorescent protein (GFP), cyan fluorescent protein, yellow fluorescent protein, orange fluorescent protein, red fluorescent protein, far-red fluorescent protein, or switchable fluorescent protein.

Prepare a Recombinant Plasmid (Named pEZ-lldR-Plldp-eGFP)

In some embodiments, a process to prepare pEZ-IldR-P_{lldp}-eGFP included:

1. Obtain pEZ15A

pEZ15A could be prepared according to “Yang S, mohaghaeghi A, franden M A, et al, Metabolic engineering of Zymomonas mobilis for 2,3-butanediol production from lignocellulosic biomass sugars [J]. Biotechnol Biofuels, 2016, 9 (1): 189”. For obtaining pEZ15A with the different coding genes (e.g. resistance genes), reference could be made to “Construction and Application of Plasmid pUC19-CM-D [J]. Agricultural Science & Technology, 2010, 11 (5): 31˜33”.

2. Prepare a Connector Named lldR-P_{Lldp}-eGFP

An operon for catabolizing lactic acid (named lldR-P_{lldp}, SEQ ID NO. 1) consisting of a transcriptin regulator (named lldR) and a lactic acid induced promoter (P_{lldp}) from Pseudomonas fluorescens A506, could be amplified with primers lldR-F (SEQ ID NO. 2) and Plldp-R (SEQ ID NO. 3). A fragment named eGFP could be amplified with primers eGFP-F (SEQ ID NO. 4) and eGFP-R (SEQ ID NO. 5). The connector lldR-P_{lldp}-eGFP (SEQ ID NO. 6) could be prepared by an Overlap PCR of lldR-P_{lldp} and eGFP.

3. Ligate with pEZ15A

A sequence of pEZ15A was reverse amplified by a PCR with primers 15Afk-F (SEQ ID NO. 7) and 15Afk-R (SEQ ID NO. 8). lldR-P_{lldp}-eGFP and the sequence of pEZ15A were ligated by a Gibson Assembly with a mole ratio of 3:1. The ligated product was transferred into competent E. coli. And positive colonies could be screened by plates containing Kanamycin. Positive colonies could further screened by colony PCR, and verified by Sequencing. As shown in FIG. 1, pEZ-IldR-P_{lldp}-eGFP could be extracted and separated from the verified positive colonies.

Therein, a reaction system of Gibson Assembly could be consisted of 0.12 pM lldR-P_{lldp}-eGFP, 0.04 pM pEZ15A, 0.5 μL 10× Buffer 4 (Thermo), 0.5 μL T5 Exonuclease, and surplus ddH₂O. And the procedure of Gibson Assembly could be processed according to Table 1.

TABLE 1

| Steps | Temperature | Time | | cycles |
|---|---|---|---|---|
| pre-denaturation | 98° C. | 3 | min | 1 |
| denaturation | 98° C. | 10 | s | 30 |
| anneal | 55° C. | 10 | s | |
| extension | 72° C. | Set 10 s/kb based on | | |
| | | the fragment length | | |
| extension | 72° C. | 2 | min | 1 |
| hold | 12° C. | 2 | min | 1 |

### Prepare a Platform Strain Named ZM4-dCas12a-Dldh

In order to test the performance of pEZ-IldR-P_{lldp}-eGFP, embodiments also prepared a platform strain named ZM4-dCas12a-Dldh. The platform strain could be applied to the screening of high D-lactic acid producing strains and the mining of genetic targets associated with high D-lactic acid production.

In some embodiments, a method for preparing ZM4-dCas12a-Dldh included: substituting the locus ZMO1650 of ZM4-dCas12a's genome with LmldhA by means of endogenous CRISPR-Cas gene editing system of the starting strain of ZM4-dCas12a; and introducing a lactic acid metabolic pathway.

Methods for preparing ZM4-dCas 12a-Dldh provided by some embodiments include:

### 1. Obtain the Starting Strain Named ZM4-dCas12a

ZM4-dCas12a could be obtained according to “Shen Wei: The Establishment and Application of the Dual Reporter-gene System and CRISPR-Cas12a Genome-editing Tool in Zymomonas mobilis [D] Hubei University; 2020”. A process to prepare ZM4-dCas12a could include site-specific mutating hydrophilic aspartic acid at the site 917 of Cas12a into hydrophobic alanine by means of the homologous recombination technology, to get an enzyme-free for DNA-cutting Cas12a. A expression box promoted with inducible promoter was constructed into the locus ZMO0038 of Z. mobilis ZM4's genome by homologous recombination.

### 2. Guilder

A 32 bp sequence of the downstream from the CCC PAM site of the gene ZMO1650 was selected as a guider (shown as SEQ ID NO:9). ZMO1650 is locate at CP023715.1:1697900 . . . 1699036 of Zymomonas mobilis subsp. mobilis ZM4=ATCC 31821 (Taxonomy ID: 264203).

### 3. Prepare a Targeting Plasmid

Some embodiments provide a targeting plasmid that carries a unit for targeting the locus ZMO1650 of Z. mobilis ZM4's genome.

In some embodiments, the method for preparing the targeting plasmid specifically included: linearizing pEZ15Asp (with the gene of spectinomycin) with restriction enzyme BsaI; annealing primers gRNA-1650-F (SEQ ID NO. 10) and gRNA-1650-R (SEQ ID NO. 11); ligating the linearized pEZ15Asp with the annealed primers by T4 ligase; transferring the ligated product into E. coli DH5a; screening positive colonies by colony PCR; and finally verifying by Sequencing. Therein, 10 μM primers were denatured at 95° C. for 5 min and then cooled to room temperature for use in the annealing process. A reaction system of the ligation may consist of 20˜40 ng linearized pEZ15Asp, 2 μL annealed primers, 0.5 μL T4 ligase, 1 μL Buffer and surplus ddH₂O. The colony PCR procedure may be set as: pre-denaturation at 98° C. for 3 min and 1 cycle; denaturation at 98° C. for 10 s, annealing at 55° C. for 10 s and extension at 72° C. (set according to fragment length for 10 s/kb for 30 cycles) for 35 cycles; and 72° C. for 5 min after the cyclic reaction, and hold at 72° C. for 2 min and 1 cycle.

### 4. Prepare an Editing Plasmid

In some embodiments, the editing plasmid could be prepared by inserting a fragment of LmldhA (SEQ ID NO. 12) into the targeting plasmid.

In some embodiment, the process to prepare the editing plasmid included: amplifying the fragment of LmldhA with primers Ldh-F (SEQ ID NO. 13) and Ldh-R (SEQ ID NO. 14) in a PCR; amplifying a upstream fragment of ZMO1650 with primers 1650US-F (SEQ ID NO. 15) and 1650US-R (SEQ ID NO. 16) in a PCR; amplifying a downstream fragment of ZMO1650 with primers 1650DS-F (SEQ ID NO. 17) and 1650DS-R (SEQ ID NO. 18) in a PCR; sequentially connecting the upstream fragment of ZMO1650, the fragment of LmldhA and the downstream fragment of ZMO1650 to get a donor (SEQ ID NO. 19); reversely amplifying the targeting plasmid with primers 15Afk-F and 15Afk-R in a PCR; ligating the fragment of the targeting plasmid and the donor in a mole ratio of 1:3 by Gibson Assembly; transferring the ligated product into E. coli DH5a; screening positive colonies by colony PCR; and finally verifying by Sequencing. Therein, Gibson Assembly and colony PCR could be executed according to above embodiments.

### 5. Prepare Competent ZM4-dCas12a

100 μL of frozen bacteria of ZM4-dCas12a were removed from a −80° C. refrigerator, and inoculated into a frozen tube with containing 1 mL RM after thawing, and static cultured in a 30° C. incubator. The culture was shifted to a 50 mL blue cap bottle filled with 20 mL RM liquid medium after it grow to logarithmic stage (OD600 nm≈1.5˜2.0), and static cultured in a 30° C. incubator. After it grow to logarithmic stage (OD600 nm≈1.5˜2.0), the culture was shifted again to a 250 mL erlenmeyer flask filled with 200 mL RM liquid medium, and enabled the initial OD600 nm in a range of 0.025˜0.05, and static cultured at 30° C., and 100 rpm in a shaker. When the culture's OD600 nm was between 0.3 and 0.4, the thalli from the culture were collected with 4000 rpm centrifugation at normal temperature, then washed with sterile water for one time and 10% glycerol for two times, finally slowly re-suspended with 400 μL 10% glycerol, and sub-packaged 50 μL into a 1.5 mL EP tube. 6. Electro-transfer p12r-1650-dldh

1 μg of p12r-1650-dldh was added to a 1.5 mL EP tube with containing 50 μL of competent ZM4-dCas12a, gently mixed and shifted into a 0.1 cm electroporation cuvette. And then the electroporation cuvette was placed into an electroporation instrument to electro-transfer. Herein, the electro-transformation conditions were set as: 200Ω, capacitor: 25 μF, voltage: 1800 V. And 1 mL RM was added into the electroporation cuvette after electro-transferring, and static cultured in a 30° C. thermostatic incubator for 4˜6 h to get the transformants. And 200 μL solution of transformants were evenously coated on a plate contained RM+Spe (with 100 μg/mL spectinomycin in RM); sealed and anastrophic incubated in a 30° C. thermostatic incubator.

### 7. Mine for ZM4-dCas12a-Dldh

After the growth of colonies, the positive colonies from these colonies could be screened by using primers 1650check-F (SEQ ID NO. 20) and 1650check-R (SEQ ID NO. 21) in colony PCR, and further verified by Sequencing. The confirmed positive colonies were named ZM4-dCas12a-Dldh.

### Mine Strains for Producing D-LA

With the help of pEZ-IldR-P_{lldp}-eGFP, high-throughput mining of metabolites could be achieved, such as converting the concentration of metabolites into fluorescence output at the single-cell level, and then high-throughput mining of microorganisms or cellular factories capable of producing these metabolites can be achieved through FACS (Fluorescence-Activated Cell Sorting).

An example tested the ability and yield of D-LA produced by the platform strain ZM4-dCas12a-Dldh constructed above. The test process of this example included:

### 1. Prepare Competent ZM4-dCas12a-Dldh

100 μL of frozen bacteria of ZM4-dCas12a-Dldh were removed from a −80° C. refrigerator, and inoculated into a frozen tube with containing 1 mL RM after thawing, and static cultured in a 30° C. incubator. The culture was shifted to a 50 mL blue cap bottle filled with 20 mL RM liquid medium after it grow to logarithmic stage (OD600 nm≈1.5˜2.0), enabled the initial OD600 nm in a range of 0.02˜0.05, and static cultured in a 30° C. incubator. After it grow to logarithmic stage (OD600 nm≈1.5˜2.0), the culture was shifted again to a 250 mL erlenmeyer flask filled with 200 mL RM liquid medium, and enabled the initial OD600 nm in a range of 0.025˜0.05, and static cultured at 30° C., and 100 rpm in a shaker. When the culture's OD600 nm was between 0.3 and 0.4, the thalli from the culture were collected at 4000 rpm centrifugation at normal temperature, then washed with sterile water for one time and 10% glycerol for two times, finally slowly re-suspended with 400 μL 10% glycerol, and sub-packaged 50 μL into a 1.5 mL EP tube.

2. Electro-Transfer pEZ-IldR-P_{Lldp}-eGFP

1 μg pEZ-IldR-P_{lldp}-eGFP was added to the bacteria solution of 50 μL competent ZM4-dCas12a-Dldh, gently mixed and shifted into a 0.1 cm electroporation cuvette. And then the electroporation cuvette was placed into an electroporation instrument to electro-transfer. Herein, the electro-transformation conditions were set as: 200Ω, capacitor: 25 μF, voltage: 1800 V. And 1 mL RM was added into the electroporation cuvette after electro-transferring, mixed well and then shifted to a sterile EP tube, and static cultured in a 30° C. thermostatic incubator for 4˜6 h to get the transformants. After colonies grew, the positive colonies from these colonies could be screened by using primers pEZ15A-F and pEZ15A-R in colony PCR, and further verified by Sequencing. The confirmed positive transformant was ZM4-dCas12a-Dldh, that had been transferred pEZ-IldR-P_{lldp}-eGFP.

### 3. Fermenting Test for ZM4-dCas12a-Dldh

ZM4-dCas12a-Dldh was fermented in an enrichment medium named RMG5 (RM, 50 g/L glucose, 10 g/L yeast extract, 2 g/L KH₂PO₄) at 30° C., 180 rpm. Samples at different time points in the fermentation process were prepared by centrifuging (12000 rpm, 2 min) to get the supernatant, and filtered the supernatant by a 0.22 m filter. Samples were tested on HPLC (LC-20 AD, Shimadzu, Japan) system consisting of Aminex HPX-87H column (300 mm×7.8 mm, Bio-Rad) and RID-20A differential refractometry detector. The concentrations of glucose, ethanol and lactic acid in fermentation liquid were detected under the following conditions that included 0.005 mol/L H₂SO₄ for mobile phase, 0.5 mL/min for flow rate, 40° C. for detector temperature, 60° C. for column temperature, and 20 μL for injection volume.

### Test Dynamic Response

Three independent colonies of ZM4-dCas12a-Dldh were randomly selected and inoculated into 3 mL RMG5 containing kanamycin as seeds. After culturing the seeds at 100 rpm in a shaker at 30° C. for 24 hours, the seeds shifted into RMG5 with the initial OD600 nm of 0.1, and cultured with gradient concentration of D-LA (0, 2, 4, 6, 8, 10 g/L). After 12 hours of culture, samples were taken for measuring the bacterial density (OD600 nm) by an ultraviolet spectrophotometer (UV-1800, Aoyi, China).

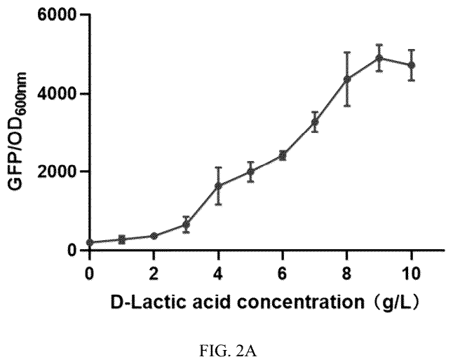

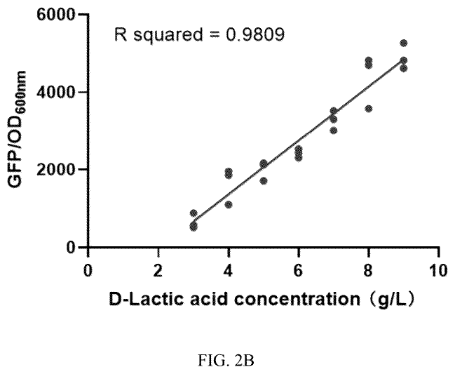

As shown in FIG. 2, the fluorescence intensity of ZM4-dCas12a-Dldh increase with the increase of D-LA concentration. The fluorescence intensity of ZM4-dCas12a-Dldh under 9 g/L lactic acid induction is 24.5 times that under no lactic acid induction. When the concentration of lactic acid is in the range of 3-9 g/L, the induced intensity of ZM4-dCas12a-Dldh is linearly positively correlated with the concentration of lactic acid, and the correlation coefficient is 0.9809.

### Mine Genes to Regulate Biosynthesis of D-LA

Embodiments provided a CRISPRi library for targeting Z. mobilis's genome.

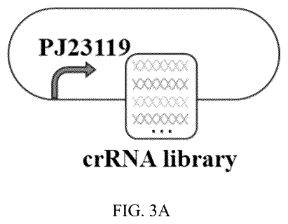

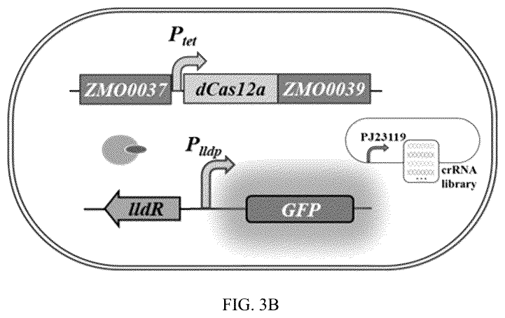

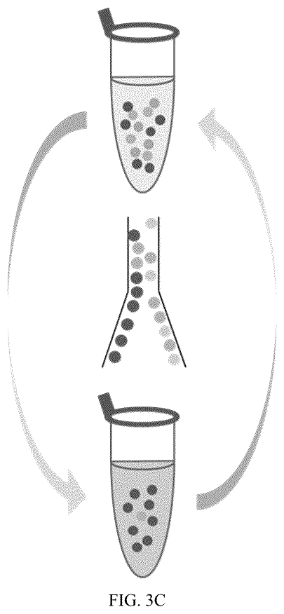

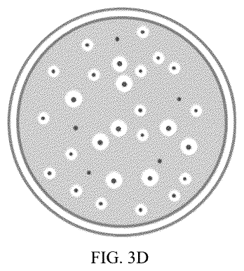

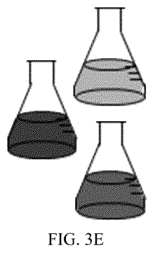

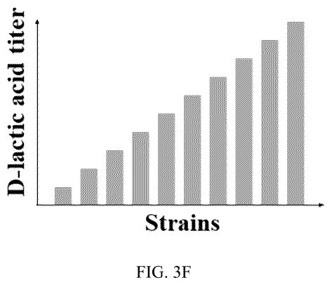

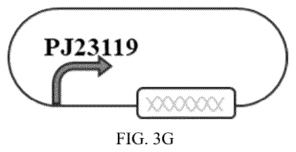

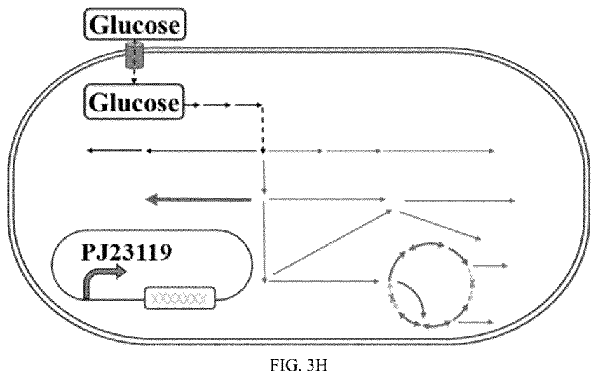

Referring to FIG. 3A, the CRISPRi library is consisting of 69,093 pEZ-sgr-lib with 69093 crRNAs, targeting 1,946 genes in the Z. mobilis genome with an average coverage of 15 crRNAs per gene. The process could refer to “Shen W., Zhang J., Geng B., et al. Establishment and application of a CRISPR-Cas 12a assisted genome editing system in Zymomonas mobilis [J]. Microb Cell Fact, 2019, 18 (1): 162”. pEZ-sgr-lib could be constructed by inserting 69093 gRNAs into pEZ-sgr, respectively. pEZ-sgr-lib was constructed and evaluated for quality control and Sequencing by GENEWIZ.CN.

As shown in FIG. 3B, pEZ-sgr-lib could be transferred into ZM4-dCas12a-Dldh, and a strain library of high throughput screening platform could be constructed by the genome-wide interference of ZM4-dCas12a-Dldh from the transformation of pEZ-sgr-lib.

As shown in FIG. 3C, mutants with strong GFP fluorescence were screened from the strain library of high throughput screening platform by two rounds of FACS (Fluorescence-activated cell sorting). A mutant library with different interference sites were obtained.

In some embodiments, the method for screening the strain library of high throughput screening platform may include:
washing the strain library twice and resuspending in 1×PBS buffer with a final strain density of 10⁷;
sorting 10⁵ strains with the stronger fluorescence intensity by MoFlo XDP (Beckman Coulter, USA) high-speed flow cytometry sorter with gates set on 510:20 nm emission filters;
excitating by a 488 nm laser, and recording Forward-scatter characteristics (FSC) as small-angle scatter and side-scatter characteristics (SSC) as orthogonal scatter of the 488 nm laser;
sorting strains with high fluorescence intensity by performing secondarily gated with the 0° detector;
cultivating the sorted strains in the test tubes and spreading on RMG5 plates with corresponding antibiotics.

As shown in FIG. 3D, the mutant library was cultivated and spread on the plate of RMG5 supplemented with calcium carbonate which high-yielding colonies were screened by assaying transparent zone on.

As shown in FIG. 3E and FIG. 3F, colonies of these mutants were further fermented by shake flasks to determine D-lactic acid yield.

As shown in FIG. 3G, the crRNA sequences in mutants with increased D-lactic acid production were obtained by Sanger Sequencing.

As shown in FIG. 3H, rational modifications were carried out on other D-lactic acid production strains based on the identified genes. And these gene could be confirmed as the gene for regulating D-LA.

Lastly, a gene named ZMO1323 was been mined for regulating D-LA by the method. And “ZMO1323” could encode histidine kinase, the ZMO1323 is locate at CP023715.1:1342357 . . . 1343994 of Zymomonas mobilis subsp. mobilis ZM4=ATCC 31821 (Taxonomy ID: 264203).

### Genes for Regulating D-LA and its Uses

In some embodiments, a mutant containing with a plasmid from pEZ-sgr-lib interference ZMO1323 was inoculated into a liquid RMG5 with no antibiotics and cultivated for 8˜16 h, and coated on a RMG5 plate with no antibiotics, respectively. By measuring the sensitivity of the mutants to the corresponding antibiotics, it was confirmed whether the pEZ-sgr-lib was dropped.

In order to further verify and explore the effect of the knockout of ZMO1323 on D-LA production, the endogenous CRISPR-Cas system was used to construct editing plasmids targeting ZMO1323. And the editing plasmids were transferred into D-LA production strain of Z. mobilis. The positive colonies were fermented to test the effect of ZMO1323 on the yield of D-LA. 1. Prepare a editing plasmid for knocking out ZMO1323

The preparing method of the editing plasmids may refer to “Characterization and repurposing of the endogenous Type I-F CRISPR-Cas system of Zymomonas mobilis for genome engineering [J], Nucleic Acids Res 2019, 47 (21): 11461-75”.

A guilder (SEQ ID NO. 22) could be designed by the 32 bp sequence of the PAM locus from the gene ZMO1323

A targeting plasmid could be prepared according the above embodiments for targeting the locus ZMO1323 of Z. mobilis ZM4's genome. The method for preparing the targeting plasmid specifically included: linearizing pEZ15Asp (with the gene of spectinomycin) with restriction enzyme BsaI; annealing primers 1323 gr-F (SEQ ID NO. 23) and 1323 gr-R (SEQ ID NO. 24); ligating the linearized pEZ15Asp with the annealed primers by T4 ligase; transferring the ligated product into E. coli DH5a; screening positive colonies by colony PCR; and finally verifying by Sequencing. Therein, 10 μM primers were denatured at 95° C. for 5 min and then cooled to room temperature for use in the annealing process. A reaction system of the ligation may consist of 20˜40 ng linearized pEZ15Asp, 2 μL annealed primers, 0.5 μL T4 ligase, 1 μL Buffer and surplus ddH₂O. The colony PCR procedure may be set as: pre-denaturation at 98° C. for 3 min and 1 cycle; denaturation at 98° C. for 10 s, annealing at 55° C. for 10 s and extension at 72° C. (set according to fragment length for 10 s/kb for 30 cycles) for 35 cycles; and 72° C. for 5 min after the cyclic reaction, and hold at 72° C. for 2 min and 1 cycle.

A US (upstream fragment of ZMO1323) was amplified with primers 1323US-F (SEQ ID NO. 25), and 1323US-R (SEQ ID NO. 26) in a PCR.

A DS (downstream fragment of ZMO1323) was amplified with primers 1323DS-F (SEQ ID NO. 27), and 1323DS-R (SEQ ID NO. 28) in a PCR.

A donor (SEQ ID NO. 29) could be prepared by connecting the US and DS.

A sequence of the targeting plasmid, which could be reversely amplified with primers 1323check-F (SEQ ID NO. 30) and 1323check-R (SEQ ID NO. 31) in a PCR, could be ligated with the donor in a mole ratio of 1:3 by Gibson Assembly. And the ligated product could be transferred into E. coli DH5a. Positive colonies could be screened by colony PCR, and finally verified by Sequencing. Therein, Gibson Assembly and colony PCR could be executed according to above embodiments. And The editing plasmid could be extracted and separated from the verified positive colonies.

2. Construct Mutants with Knockout of ZMO1323

The mutants with knockout of ZMO1323 could be constructed by transferring the editing plasmids into different D-LA production strain of Z. mobilis. The constructing method refers to above embodiments.

The D-LA production strain of Z. mobilis could select from ZMNP-HAPL, ZML-pdc-ldh (CN115851569A).

ZMNP-HAPL could be constructed with substitution of ZMO0038, ZMO1650 and ZMO1360 of ZMNP-HAP with LmldhA referring to CN115851569A. ZMNP-HAP could be prepared referring to “Shen Wei, Unravel molecular mechanisms of lactate tolerance for lactate-production strain construction in Zymomonas mobilis [D] Hubei University, 2020”.

By the knockout of ZMO1323, mutants could be named ZMNP-HAPLA1323, ZML-pdc-1dhA1323.

### 3. Fermentation Test of Mutants

These mutants could be performed with fermentation test. These mutants were inoculated into RMG5 with 80% of bottling quantity and 100 g/L calcium carbonate in 50 mL erlenmeyer flasks, respectively, cultivated at 100 rpm, and at 30° C. in a shaker. At the fermentation process, samples could be made by the supernatant from centrifugation (12000 rpm, 2 min) for HPLC. The liquid RMG5 included 50 g/L glucose, 10 g/L yeast extract and 2 g/L KH₂PO₄.

### 4. HPLC

The contents of glucose, lactic acid, or ethanol in the samples could be tested by a HPLC system that consists of Prominence Plus HPLC (LC-20AD, Shimadzu (Japan)), Differential refraction Detector (RID, RID-20A), column (Bio-Rad Aminex HPX-87H, 300 mm×47.8 mm), 5 mM of sulfuric acid used as the mobile phase. The temperature of Detection cell of the HPLC is 40° C., and the temperature of column is 60° C. The flow rate of mobile phase is 0.5 mL/min. Samples could be filtered by 0.22 μm filters, and 20 μL volume for injection.

The optical purity of D-LA in samples could be tested by another HPLC system that consists of: Agilent 1260 HPLC (1260 Infinity II, Agilent (USA)), Differential refraction Detector (RID, RID-20A), PDA (photo-diode array, 1260 Infinity II HS, Agilent (USA)), chiral column (Chirex® 3126 (D)-penicillamine, LC Column 250×4.6 mm, Ea, 00G-3126-E0, CHIREX), 5% isopropyl alcohol solution of 2 mmol/L CuSO₄ used as the mobile phase. The temperature of Detection cell of the HPLC is 30° C., and the temperature of column is 30° C. The flow rate of mobile phase is 1.0 mL/min. Injection volume is 5 μL. Samples could be filtered by 0.22 μm filters for injection.

TABLE 2
HPLC test results of mutants

| Strains and Mutants | C_{glu}(g/L) | Ethanol (g/L) | Yield (g/L) | V_{LA}(g/(L · h)) | η_{c}(%) |
|---|---|---|---|---|---|
| ZMNP-HAPL | 80.99 ± 0.01 | 26.05 ± 0.03 | 27.71 ± 0.05 | 2.03 ± 0.02 | 98.6 ± 0.00 |
| ZMNP-HAPLΔ1323 | 81.92 ± 0.01 | 24.33 ± 0.02 | 31.85 ± 0.01 | 2.11 ± 0.01 | 98.3 ± 0.00 |
| ZML-pdc-ldh | 82.13 | 27.33 ± 0.03 | 26.01 ± 0.05 | 2.01 ± 0.02 | 98.2 ± 0.00 |
| ZML-pdc-ldhΔ1323 | 82.09 | 25.12 ± 0.01 | 31.57 ± 0.02 | 2.09 ± 0.01 | 99.7 ± 0.05 |

In the Table 2, “C_{glu}” refers to the consumption of Glucose and could be calculated by the difference value of its content in fermentation broth at the beginning and the end of fermentation.

In the Table 2, “Ethanol” refers to the ethanol content at the end of fermentation broth.

In the Table 2, “V_{LA}” refers to production rate of D-LA and could be calculated by formula: V_{LA}=d(g_{LA})/dt. Therein, “d(g_{LA})” refers to the yields of D-LA in different time of fermentation, “dt” refers to the time spent of fermentation.

In the Table 2, “Yield” refers to the yield of D-LA at the end of fermentation.

In the Table 2, “η_{c}” refers to the carbon conversion rata and could be calculated by formula: η_{c}=(g_{LA}/1+gₑₜₕₐₙₒₗ/0.511)/C_{glu}. Therein, “g_{LA}” and “gₑₜₕₐₙₒₗ” refer to the yields of D-LA and ethanol, respectively. “1” and “0.511” refer to the theoretical value of the Glucose conversion to D-LA and ethanol, respectively.

As shown in Table 2, the mutants with the knockout of ZMO1323 have a gain of the yield of D-LA.

The above is only the preferred embodiments of this disclosure and is not intended to limit this disclosure. Any modification, equivalent replacement, improvement, etc., made within the spirit and principle of this disclosure shall be included in the scope of this disclosure.

What is claimed is:

1. A method for mining strains to produce D-LA comprising:
obtaining a recombinant plasmid, the recombinant plasmid carries a connector consisting of a operon for catabolizing D-lactic acid and a gene for report, the operon for catabolizing D-lactic acid is shown as SEQ ID NO. 1;
transferring the recombinant plasmid into a strain to be mined;
screening positive colonies according to the fluorescence intensity of the strain to be mined.

2. The method according to claim 1 wherein, the method comprising a first process to prepare the the recombinant plasmid, the first process comprising:
preparing a plasmid named pEZ15A;
inserting the operon and the gene for report into the pEZ15A.

3. The method according to claim 1 wherein, the gene for report is selected from the group consisting of genes of firefly luciferase, marine coelenteric luciferase, secretory alkaline phosphatase, human growth hormone, green fluorescent protein, cyan fluorescent protein, yellow fluorescent protein, orange fluorescent protein, red fluorescent protein, far-red fluorescent protein, or switchable fluorescent protein.

4. The method according to claim 1 wherein, the method comprising a second process to prepare the strain to be mined, the second process comprising:
preparing a strain named ZM4-dCas12a;
substituting a locus ZMO1650 of ZM4-dCas12a's genome with a gene named LmldhA, the ZMO1650 is locate at CP023715.1:1697900 . . . 1699036 of Zymomonas mobilis subsp. mobilis ZM4=ATCC 31821.

5. The method according to claim 1 wherein, the connector is named lldR-P_{lldp}-eGFP shown as SEQ ID NO. 6.

6. A method for mining genes to regulate biosynthesis of D-lactic acid comprising:
obtaining a recombinant plasmid, the recombinant plasmid carries a connector consisting of a operon for catabolizing D-lactic acid and a gene for report, the operon for catabolizing D-lactic acid is shown as SEQ ID NO. 1;
transferring the recombinant plasmid into a strain to be mined;
screening positive colonies according to the fluorescence intensity of the strain to be mined; and
mining the genes to regulate biosynthesis of D-lactic acid from the positive colonies.

7. The method according to claim 6 wherein, the method comprising a first process to prepare the the recombinant plasmid, the first process comprising:
preparing a plasmid named pEZ15A;
inserting the operon and the gene for report into the pEZ15A.

8. The method according to claim 6 wherein, the gene for report is selected from the group consisting of genes of firefly luciferase, marine coelenteric luciferase, secretory alkaline phosphatase, human growth hormone, green fluorescent protein, cyan fluorescent protein, yellow fluorescent protein, orange fluorescent protein, red fluorescent protein, far-red fluorescent protein, or switchable fluorescent protein.

9. The method according to claim 6 wherein, the connector is named lldR-P_{lldp}-eGFP shown as SEQ ID NO. 6.

10. The method according to claim 6 wherein, the method comprising a second process to prepare the strain to be mined, the second process comprising:
preparing a strain named ZM4-dCas12a;
substituting a locus ZMO1650 of ZM4-dCas12a's genome with a gene named LmldhA, the ZMO1650 is locate at CP023715.1:1697900 . . . 1699036 of Zymomonas mobilis subsp. mobilis ZM4=ATCC 31821.

11. The method according to claim 6 wherein, said gene to regulate biosynthesis of D-lactic acid is ZMO1323, the ZMO1323 is locate at CP023715.1:1342357 . . . 1343994 of Zymomonas mobilis subsp. mobilis ZM4=ATCC 31821.

12. A use of ZMO1323 in biosynthesis of D-lactic acid comprising:
preparing an editing plasmid for knocking out the ZMO1323, the ZMO1323 is locate at CP023715.1:1342357 . . . 1343994 of Zymomonas mobilis subsp. mobilis ZM4=ATCC 31821;
transferring the editing plasmid into a D-lactic acid production strain of Zymomonas mobilis.

13. The use according to claim 12 wherein, the step of preparing the editing plasmid for knocking out the ZMO1323 comprising:
preparing a guilder by annealing primers 1323 gr-F shown as SEQ ID NO. 23, and 1323 gr-R shown as SEQ ID NO. 24;
inserting the guilder into a plasmid named pEZ15Asp to prepare a targeting plasmid;
preparing a donor shown as SEQ ID NO. 29;
inserting the donor into the targeting plasmid.

14. The use according to claim 13 wherein, a step of preparing the pEZ15Asp comprising:
preparing a named pEZ15A;
insert a gene of spectinomycin into the pEZ15A.

15. The use according to claim 13 wherein, a step of inserting the guilder into a plasmid pEZ15Asp to prepare a targeting plasmid comprising:
linearizing the plasmid pEZ15Asp with restriction enzyme BsaI;
ligating a linearized pEZ15Asp with the guilder by T4 ligase;
transferring a ligated product into E. coli DH5a;
screening positive colonies by a colony PCR; and
verifying by sequencing.

16. The use according to claim 13 wherein, the upstream fragment of ZMO1323 is amplified with primes 1323US-F and 1323US-R in a PCR;
the downstream fragment of ZMO1323 is amplified with primes 1323DS-F and 1323DS-R in a PCR;
the 1323US-F is shown as SEQ ID NO. 25, the 1323US-R is shown as SEQ ID NO. 26;
the 1323DS-F is shown as SEQ ID NO. 28, the 1323DS-R is shown as SEQ ID NO. 28.

17. The use according to claim 15 wherein, a reaction system of the ligation comprising: 20˜40 ng linearized pEZ15Asp, 2 μL annealed primers, 0.5 μL T4 ligase, 1 μL Buffer and surplus ddH₂O.

18. The use according to claim 13 wherein, a step of inserting the donor into the targeting plasmid comprising:
ligating a fragment of the targeting plasmid and the donor in a mole ratio of 1:3 by a gibson assembly.

19. The use according to claim 18 wherein, a step of preparing the fragment of the targeting plasmid comprising:
reversely amplifying the targeting plasmid with primers 15Afk-F shown as SEQ ID NO. 7 and 15Afk-R shown as SEQ ID NO. 8 in a PCR.

20. The use according to claim 12, the D-lactic acid production strain of Zymomonas mobilis is selected from the group consisting of: a strain named ZMNP-HAPL, or a strain named ZML-pdc-ldh.


SEQUENCE LISTING (31 sequences)

SEQ ID NO: 1 - DNA, 1019 bp, synthetic construct
  source 1..1019: mol_type = other DNA
ctacatccctttgcgtcgctccgcccgagccacgcgttgaacctcctgacgcacctcctccaacacctcctgcacatacagcagatgccgtgtcgagacttcgcgcgcctgctcggcccgcccctcgatgatggccaggtacaagtcccgatgctgattgatcagcatgtcgcgggtctcggtacgctgctggtacatgccaccaatattcgtcaccacgttacgcttgagcaggtcgaacagcccgcgaatggtgtgcagcaacacggcgttatgactggcttcggcaatcgccaagtgaaaccttgcatccgcagcaccctcctccactcggctcacttcgtcggcgcgcgcatagcaatcctgcaacgcttcaaacgctgcggtcagccgttcacggtcaacctccgtggcgcgtaacgccgcgtaataagcacacgaagcctcaagggtctggcgaaactccagcagatcgcgctgcgcttcgggattgctttccaacaagtgcaacagcggatcgctgaaggtcgaacccagggagtccaccacatagttgccaccgccctgacgactgaccaacaaccccttggccgccagtttctgaatcgcctcacgcaacgacgggcgcgacacaccaaagcgctcggctaacgcgcgctcggccggcaagcgttcgcccgacttcagcgtgccctcgagaatcatcccctcaagctgctcgacaatatcgtcagacaaacggcgctgacgcacctgatcaaaccccataactcactctccaccatcccgaccactcgccggggcccctattctggcctatcgacgtgccggcagcacctatcagaacgccttcaatttcgccgatcagaacgccccgcctaccgacctacgaccaaagtttcgcaggcggcaaattgacacacccctgcccaggctcttaatctagcccacagcgactgtaaattggtattaccaattaaccaagctcaccaaacaacaacaatcaggggccaccccat

SEQ ID NO: 2 - DNA, 35 bp, synthetic construct
  source 1..35: mol_type = other DNA
atcagatctctcgagctacatccctttgcgtcgct

SEQ ID NO: 3 - DNA, 30 bp, synthetic construct
  source 1..30: mol_type = other DNA
gcccttgctcaccatatggggtggcccctg

SEQ ID NO: 4 - DNA, 33 bp, synthetic construct
  source 1..33: mol_type = other DNA
caggggccaccccatatggtgagcaagggcgag

SEQ ID NO: 5 - DNA, 31 bp, synthetic construct
  source 1..31: mol_type = other DNA
tttatacagttcatccatgcccagatcaatg

SEQ ID NO: 6 - DNA, 1739 bp, synthetic construct
  source 1..1739: mol_type = other DNA
ctacatccctttgcgtcgctccgcccgagccacgcgttgaacctcctgacgcacctcctccaacacctcctgcacatacagcagatgccgtgtcgagacttcgcgcgcctgctcggcccgcccctcgatgatggccaggtacaagtcccgatgctgattgatcagcatgtcgcgggtctcggtacgctgctggtacatgccaccaatattcgtcaccacgttacgcttgagcaggtcgaacagcccgcgaatggtgtgcagcaacacggcgttatgactggcttcggcaatcgccaagtgaaaccttgcatccgcagcaccctcctccactcggctcacttcgtcggcgcgcgcatagcaatcctgcaacgcttcaaacgctgcggtcagccgttcacggtcaacctccgtggcgcgtaacgccgcgtaataagcacacgaagcctcaagggtctggcgaaactccagcagatcgcgctgcgcttcgggattgctttccaacaagtgcaacagcggatcgctgaaggtcgaacccagggagtccaccacatagttgccaccgccctgacgactgaccaacaaccccttggccgccagtttctgaatcgcctcacgcaacgacgggcgcgacacaccaaagcgctcggctaacgcgcgctcggccggcaagcgttcgcccgacttcagcgtgccctcgagaatcatcccctcaagctgctcgacaatatcgtcagacaaacggcgctgacgcacctgatcaaaccccataactcactctccaccatcccgaccactcgccggggcccctattctggcctatcgacgtgccggcagcacctatcagaacgccttcaatttcgccgatcagaacgccccgcctaccgacctacgaccaaagtttcgcaggcggcaaattgacacacccctgcccaggctcttaatctagcccacagcgactgtaaattggtattaccaattaaccaagctcaccaaacaacaacaatcaggggccaccccatatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcaccgccgacaagcagaagaacggcatcaaggccgaatttgaaattcgtcataatgtggaagatggcagcgtgcagctggcggatcattatcagcagaataccccgattggcgatggcccagtgctgctgccggatgaccactatctgagcaccgaaagcgtgctgagcaaagatccgaatgaagatcgtgatcatatggtcctgctggaatttgtgaccgcggcaggcattgatctgggcatggatgaactgtataaataa

SEQ ID NO: 7 - DNA, 31 bp, synthetic construct
  source 1..31: mol_type = other DNA
agtgatatcagatctcgagctcggtacccgg

SEQ ID NO: 8 - DNA, 20 bp, synthetic construct
  source 1..20: mol_type = other DNA
agacggtgagctggtgacct

SEQ ID NO: 9 - RNA, 32 nt, synthetic construct
  source 1..32: mol_type = other RNA
ttcaaaagaagtattggtaagcgagaccacgg

SEQ ID NO: 10 - DNA, 36 bp, synthetic construct
  source 1..36: mol_type = other DNA
gaaattcaaaagaagtattggtaagcgagaccacgg

SEQ ID NO: 11 - DNA, 36 bp, synthetic construct
  source 1..36: mol_type = other DNA
gaacccgtggtctcgcttaccaatacttcttttgaa

SEQ ID NO: 12 - DNA, 1301 bp, synthetic construct
  source 1..1301: mol_type = other DNA
gttcgatcaacaacccgaatcctatcgtaatgatgttttgcccgatcagcctcaatcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatatagtagctacttaataagttaggagaataaacatgaagatttttgcttacggcattcgtgatgatgaaaagccatcacttgaagaatggaaagcggctaacccagagattgaagtggactacacacaagagctattgacacctgaaacagttaagttggctgagggatcagattcagctgttgtttaccaacaactggactatacacgtgaaacattgacagctttagctaacgttggtgttactaacttgtcattgcgtaacgttggtacagataacattgattttgatgcagcacgtgaatttaactttaacatttcaaatgttcctgtttattcaccaaatgctattgcagaacactcaatgattcaattatctcgtttgctacgtcgcacgaaagcattggatgccaaaattgctaagcacgacttgcgctgggcaccaacaattggacgtgaaatgcgtatgcaaacagttggtgttattggtacaggccatattggccgtgttgctattaacattttgaaaggctttggggcaaaggttattgcttatgataagtacccaaatgctgaattgcaagcagaaggtttgtacgttgacacattagacgaattatatgcacaagctgatgcaatttcattgtatgttcctggtgtgcctgaaaaccatcatctaatcaatgcagaggctattgctaagatgaaggatggcgtggttatcatgaatgctgcgcgtggtaatttgatggacattgatgctattattgatggtttgaattctggtaagatttcagacttcggtatggacgtttatgaaaatgaagttggcttgttcaatgaagattggtctggtaaagaattcccagatgctaagattgctgacttgatttcacgcgaaaatgtattggttacgccacatacggctttctatacaactaaagctgttctagaaatggttcaccaatcatttgatgcagcagttgctttcgccaaaggtgagaagccagctattgctgttgaatattaa

SEQ ID NO: 13 - DNA, 35 bp, synthetic construct
  source 1..35: mol_type = other DNA
ttcataatttgcataagtcttgatgtaaaaaatac

SEQ ID NO: 14 - DNA, 49 bp, synthetic construct
  source 1..49: mol_type = other DNA
ctaatcaagaccgcaccttaatattcaacagcaatagctggcttctcac

SEQ ID NO: 15 - DNA, 37 bp, synthetic construct
  source 1..37: mol_type = other DNA
gtcaccagctcaccgtctccgatccgccctatggtct

SEQ ID NO: 16 - DNA, 32 bp, synthetic construct
  source 1..32: mol_type = other DNA
ggaggatattccagagaagaaagtaagcaatc

SEQ ID NO: 17 - DNA, 24 bp, synthetic construct
  source 1..24: mol_type = other DNA
ggtgcggtcttgattagccttgaa

SEQ ID NO: 18 - DNA, 38 bp, synthetic construct
  source 1..38: mol_type = other DNA
tcgagatctgatatcactgtgctatccgcttggctctc

SEQ ID NO: 19 - DNA, 2846 bp, synthetic construct
  source 1..2846: mol_type = other DNA
ccgatccgccctatggtctgaccaatcttgatgaatccgacatcaaagagatgaactacaatggtgtgttccgtctctctcccgatggtcgtttagatttgatcgaagctggtcttagccgcccgaatggtttggctctgtccccggatgaaacgaaactttatgtttctaactcagaccgcgccagcccgaatatctgggtttattctttggatagcaatggcttgccaaccagccggactttgctgcggaacttccgcaaggaatatttcgatcagggtctcgccggtcttcctgatggtatgaatatcgacaaacagggcaatctttttgcttctgctccgggcggtatctatatttttgcaccagatggcgaatgcttgggtcttatttctggtaacccaggtcagccgttgtcgaactgctgctttggtgaaaaaggccagaccttgttcatctcggcttctcacaatgttgtgcgcgttcgcaccaaaacctttggataattattgtctgtcataaaaatataaaaaacagataatatacaaaattcaggcccgttttaagggaagtaatagaagaggatgccaaaaggcaaattcctgacgggccctgataagagaaagaacaaaagaattctttcttaagaatactctataatttggctatctttcttttctgaccgctttattgtcggaggatattccagagaagaaagtaagcaatcttcataatttgcataagtcttgatgtaaaaaatacatatttagaaagaacaagcagccttgctcatcaccgctgtcgcgagtagaaaaatctcggctttcagaaaatagaggtcgcttcgttaaacagactataaatgtgctggaataaagcgaaccccttgatctgataaaactgatagacatattgcttttgcgctgcccgattgctgaaaatgcgtaaaattggtgattttactcgttttcaggaaaaactttgagaaaacgtctcgaaaacgggattaaaacgcaaaaacaatagaaagcgatttctcgaaaatggttgttttcgggttgttgctttaaactagtatgtagggtgaggttatagctatgaagatttttgcttacggcattcgtgatgatgaaaagccatcacttgaagaatggaaagcggctaacccagagattgaagtggactacacacaagagctattgacacctgaaacagttaagttggctgagggatcagattcagctgttgtttaccaacaactggactatacacgtgaaacattgacagctttagctaacgttggtgttactaacttgtcattgcgtaacgttggtacagataacattgattttgatgcagcacgtgaatttaactttaacatttcaaatgttcctgtttattcaccaaatgctattgcagaacactcaatgattcaattatctcgtttgctacgtcgcacgaaagcattggatgccaaaattgctaagcacgacttgcgctgggcaccaacaattggacgtgaaatgcgtatgcaaacagttggtgttattggtacaggccatattggccgtgttgctattaacattttgaaaggctttggggcaaaggttattgcttatgataagtacccaaatgctgaattgcaagcagaaggtttgtacgttgacacattagacgaattatatgcacaagctgatgcaatttcattgtatgttcctggtgtgcctgaaaaccatcatctaatcaatgcagaggctattgctaagatgaaggatggcgtggttatcatgaatgctgcgcgtggtaatttgatggacattgatgctattattgatggtttgaattctggtaagatttcagacttcggtatggacgtttatgaaaatgaagttggcttgttcaatgaagattggtctggtaaagaattcccagatgctaagattgctgacttgatttcacgcgaaaatgtattggttacgccacatacggctttctatacaactaaagctgttctagaaatggttcaccaatcatttgatgcagcagttgctttcgccaaaggtgagaagccagctattgctgttgaatattaaggtgcggtcttgattagccttgaagcctgacttcaaggctaagagaaaaaatagattttagataaagacaaccaaacatcctaagaataggatgttaatttataaccacgccgtgtttttttgcccataattcaagctggtcaccaatattgctggcaccgcttgcaaccaattcccgaataaagggcgcaaaagcatcccgatccaaagagcggatcattgccttgataggccctatcgccactggtgtgatagaaagccgttttaagcccaatccgatgagtgccatggccgtcagagtacgaccacccatttccccgcaaatgccgaccttacaacccgcagcattggcctgatcgataatccgtgacaagaaacgcagcaaagccggagagagccagtcatatctttctgccagcttgggataagcacgatcggctgcaaaaaagaactggatcagatcattagtgccaatcgacaagaaatcgagcttgggcaacagaatatcaagcgtctcggctaaggaaggcacctctagcatcgagccataataaattctcttcgcgcgcggtttgtcatgtttggcaagccaagcattttgcttttcaaagacagcgcgagcctgttcatattcccatggctcggcaatcatcgggaacatgacataaagcgtttttcccgcagccccttcaatcaatgcccgtgcctgaaccttgagcagggctttcttttcgagagccaagcggatagcac

SEQ ID NO: 20 - DNA, 21 bp, synthetic construct
  source 1..21: mol_type = other DNA
ccgatggcaaaatctgggttg

SEQ ID NO: 21 - DNA, 20 bp, synthetic construct
  source 1..20: mol_type = other DNA
taccgagacgggaaagacag

SEQ ID NO: 22 - RNA, 32 nt, synthetic construct
  source 1..32: mol_type = other RNA
gctatcactggcggagaaacgagcttgcgctt

SEQ ID NO: 23 - DNA, 36 bp, synthetic construct
  source 1..36: mol_type = other DNA
gaaagctatcactggcggagaaacgagcttgcgctt

SEQ ID NO: 24 - DNA, 36 bp, synthetic construct
  source 1..36: mol_type = other DNA
gaacaagcgcaagctcgtttctccgccagtgatagc

SEQ ID NO: 25 - DNA, 36 bp, synthetic construct
  source 1..36: mol_type = other DNA
ccagctcaccgtctgatgactgcttcaatcgccttg

SEQ ID NO: 26 - DNA, 32 bp, synthetic construct
  source 1..32: mol_type = other DNA
tccattctttattcttcggcaaatcggtagcc

SEQ ID NO: 27 - DNA, 43 bp, synthetic construct
  source 1..43: mol_type = other DNA
gccgaagaataaagaatggataaaatagctctgacattccaag

SEQ ID NO: 28 - DNA, 35 bp, synthetic construct
  source 1..35: mol_type = other DNA
agatctgatatcacttcgggtgcggctatcaatac

SEQ ID NO: 29 - DNA, 1610 bp, synthetic construct
  source 1..1610: mol_type = other DNA
atgactgcttcaatcgccttggtcgatgatgaccgtaatattttagcctcggtttcgatgacgcttcagtcagagggattttctgtcagagtctattctgatgggaaaacggctctgaatgcgcttttggaatcgccgccggatttggttgttttggatatcaaaatgcccatcatggacggaatggaggttttgcggcgattgcgtgaaaaaagcatggtgccggtcatttttctgacctccaaagatgatgagctggatgaggccttgggtttggcttctggtgccgatgattatatcgccaagcctttctctttgcggctgttaatcgcgcggattagggccttgttacggcgcaccgaagccagatttcaggaaaaatctgaagaaacgccccaaaatagccatatcattgttagaggtcgtctggtgatggaccctgatcggcatcaggtgtcatgggacggtgaggatatttccttgacggtgacagagtttctgatccttgaggccttggcgcagcgtcccggttttgtaaaatcgcgtgaccaattgatggataccgcctatcaggatgatgtctatgtcgatgatcgcaccatagacagccatatcaagcgcctgcggcgtaaattccgccaggtagataatcaattcaaaagtatagagacgctctatggtatcggctaccgatttgccgaagaataaagaatggataaaatagctctgacattccaagcgaaatggatgaaaatttccaccgaaaaaggtgattttggaatatctatttcttatcgcttttgactgtgattatctttgtaatgtcatgggctaaagcgttctaattgagatgtcttctgtaactattgaaaatacccgcaaggccggtggttcaaacgaatcatcggccttgattcacgcaacaacgatattcatcaaaaatcacggcgttcttttgatcggtgcgtctggtagcgggaaatccgaccttgctttgcgcctgatagatcgcggagccattctggtcagtgatgattacagcttgatagaagatgatcccgaaaaacggcttttggttgcctctccgccttctcatattgcgggtttattagaaataagaggtgtggggattgttaagatgcccttttcgcctaaggcgaatctggctttggctgtcattttggatgacagcgttgagcggatgcctgatcctgcccattctgtagaaattttagggcgggctttgccgcttttaacgcttaaggcttttgaggcttcgacaccgataaaaattgaaatgaaaatagcgtgtttacagggagatattctgctaaaagattgagattgaagaggatatcttacctatgagccggaatgatttgtcttctgccccctcgtcttctgcggctccgcctgcgcgtattttattggtaaccggattgtcgggtgccggaaaatcaacagcgcttcgcacttttgaagatatgggatgggaaaccgtcgataatttgccgctctccctgttggagcggttaattcttacgccgccgtcatcggtcgcggcctataaagggcggcctttggctttgggtattgatagccgcacccga

SEQ ID NO: 30 - DNA, 20 bp, synthetic construct
  source 1..20: mol_type = other DNA
caagtcaccggaagtccgaa

SEQ ID NO: 31 - DNA, 20 bp, synthetic construct
  source 1..20: mol_type = other DNA
gtgccaaaaatgcccgttct